(19) United States
(12) Patent Application Publication
(10) Pub. No.: US 2014/0118687 A1
(43) Pub. Date: May 1, 2014

(54) OPHTHALMIC APPARATUS AND CONTROL METHOD THEREFOR

(71) Applicant: CANON KABUSHIKI KAISHA, Tokyo (JP)

(72) Inventors: Hideyuki Ohban, Kawaguchi-shi (JP); Satoshi Aikawa, Yokohama-shi (JP); Yoshitaka Nakano, Kawasaki-shi (JP); Yohei Saito, Chigasaki-shi (JP)

(73) Assignee: CANON KABUSHIKI KAISHA, Tokyo (JP)

(21) Appl. No.: 14/050,497

(22) Filed: Oct. 10, 2013

(30) Foreign Application Priority Data
Oct. 26, 2012 (JP) 2012-237265

(51) Int. Cl. A61B 3/12

(52) U.S. Cl. CPC A61B 3/12
USPC 351/206; 351/246

(57) ABSTRACT

An ophthalmic apparatus includes an illumination optical system which projects an illumination light beam from an illumination light source onto the fundus of the eye to be examined and an imaging optical system which guides reflected light from the fundus to imaging part. The ophthalmic apparatus calculates the contrast value of the fundus image formed by the imaging part, and focuses the imaging optical system on the fundus by moving a focus lens in the optical-axis direction of the imaging optical system based on the contrast value obtained by the calculation. The apparatus adjusts the contrast value obtained by the above calculation based on the position of the focus lens in the optical-axis direction.

### BACKGROUND OF THE INVENTION

1. Field of the Invention

The present invention relates to an ophthalmic apparatus and a control method therefor.

2. Description of the Related Art

In general, a fundus camera projects split focus indices on the pupil of an eye to be examined to facilitate detecting a focal position on the fundus of the eye at the time of focusing operation. The operator performs focusing while observing a positional relationship between the reflected focus index images through a focus lens disposed in an imaging optical system. It is also known to image the focus index images projected and reflected by the eye and perform autofocus operation from the positional relationship between the focus index images.

However, only setting the focus index images in a predetermined positional relationship (aligning them in a line) will cause an error in a detected focal position on the fundus of the eye to be examined due to the influence of aberration in the optical system inside the eye due to astigmatism or the like. This makes it difficult to perform imaging at an accurate focal position.

In order to solve the above problem, Japanese Patent Laid-Open No. 2011-50531 (to be referred to as literature 1 hereinafter) discloses a fundus camera which performs focusing by directly using a specific region of the fundus of the eye to be examined for focal position detection without using any focus index images for focal position detection with respect to the fundus of the eye. The fundus camera proposed in literature 1 performs autofocus operation by detecting the contrast of a specific region on the fundus of the eye to be examined by a focusing state detection part, calculating a contrast value based on the detection result, and determining a position where the contrast value is maximal as a focal position. In addition, the fundus camera disclosed in literature 1 includes an illumination light amount control part which adjusts the amount of illumination light emitted by an observation light source as a technique for implementing accurate autofocus operation. When, for example, detecting a contrast at a papillary portion where highlight detail loss tends to occur, this illumination light amount control part detects the contrast first by using a focusing state detection part and then controls the illumination light amount based on the detection result, thereby performing autofocus operation.

However, when using a non-mydriatic fundus camera designed to perform observation by using infrared light, since the contrast at a specific region of the fundus of the eye to be examined is low relative to infrared light, it is difficult to detect a maximal point of contrast values which corresponds to the position of a focus lens. A camera disclosed in Japanese Patent Laid-Open No. 5-199998 (to be referred to as literature 2 hereinafter) is provided with a reflected light amount detection part which detects the amount of light reflected by the fundus of the eye to be examined to sharpen a reflection image of infrared light from the fundus of the eye. The fundus camera disclosed in literature 2 operates a contrast enhancement and edge enhancement means in accordance with the amount of light detected by this reflection light amount detection part.

The luminance value of a fundus image of the eye to be examined which is captured by an image sensor changes depending on the position of a focus lens disposed in an imaging optical system. This is because as the position of the focus lens changes, the imaging magnification (field angle) of the fundus image captured by the image sensor changes to result in a change in illuminance on the image sensor. Compare, for example, a case in which the focus lens is located on the myopic side with a case in which the focus lens is located on the hyperopic side. The imaging magnification on the hyperopic side is larger, and hence the illuminance on the image sensor is lower, resulting in smaller luminance values. In general, the luminance value of a fundus image of the eye to be examined has an influence on a contrast value. For this reason, when determining, as a focal position, a position where the contrast value of a specific region of the fundus of the eye to be examined becomes maximal, if the luminance value of a fundus image of the eye is low or varies depending on the position of the focus lens, it is difficult to detect an accurate focal position.

The fundus camera disclosed in literature 1 calculates a contrast value at a specific region of the fundus of the eye to be examined with a constant illumination light amount under control, and hence still has the above problem. In addition, with regard to the fundus camera disclosed in literature 2, there are no descriptions about an arrangement for coping with a luminance value which changes depending on the position of the focus lens and an autofocus process using a contrast value.

### SUMMARY OF THE INVENTION

An embodiment of the present specification provides an ophthalmic apparatus which implements accurate autofocus in autofocus operation performed by using the contrast value of a fundus image which is obtained by illuminating the fundus of the eye to be examined and a control method for the apparatus.

According to one aspect of the present invention, there is provided an ophthalmic apparatus comprising: an illumination optical system which projects an illumination light beam from an illumination light source onto a fundus of an eye to be examined; an imaging optical system which guides reflected light from the fundus to imaging unit; a calculation unit configured to calculate a contrast value of a fundus image formed by the imaging unit; a focusing unit configured to focus the imaging optical system on the fundus by moving a focus lens in an optical-axis direction of the imaging optical system based on a contrast value obtained by the calculating unit; and an adjusting unit configured to adjust the contrast value obtained by the calculating unit based on a position of the focus lens in the optical-axis direction.

Also, according to another aspect of the present invention, there is provided a control method for an ophthalmic apparatus including an illumination optical system which projects an illumination light beam from an illumination light source onto a fundus of an eye to be examined and an imaging optical system which guides reflected light from the fundus to imaging unit, the method comprising: a calculation step of calculating a contrast value of a fundus image formed by the imaging unit; a focusing step of focusing the imaging optical system on the fundus by moving a focus lens in an optical-axis direction of the imaging optical system based on a contrast value obtained in the calculation step; and an adjusting step of adjusting the contrast value obtained in the calculation step based on a position of the focus lens in the optical-axis direction.

Further features of the present invention will become apparent from the following description of exemplary embodiments with reference to the attached drawings.

### BRIEF DESCRIPTION OF THE DRAWINGS

FIG. 1 is a view showing an example of the schematic arrangement of a fundus camera according to the first embodiment;

FIG. 2 is a block diagram showing an example of the control arrangement of the fundus camera according to the first embodiment;

FIG. 3 is a view showing an example of the fundus image of the eye to be examined which is displayed on a monitor and a focus detection range;

FIG. 4A is a view showing the schematic arrangement of a focus detection part;

FIG. 4B is a graph for explaining an example of the principle of contrast detection;

FIG. 5A is a view showing an example of the schematic arrangement of an emitted light amount calculating part;

FIG. 5B is a graph showing an example of a concept of contrast value transition in infrared light;

FIG. 5C is a graph showing an example of a concept of luminance value transition on an image sensor 31 corresponding to a focus lens position;

FIG. 6 is a flowchart showing an example of autofocus in the first embodiment;

FIG. 7 is a view showing an example of the schematic arrangement of a fundus camera according to the second embodiment;

FIG. 8 is a block diagram showing an example of the control arrangement of the fundus camera according to the second embodiment; and

FIG. 9 is a flowchart showing an example of autofocus according to the second embodiment.

### DESCRIPTION OF THE EMBODIMENTS

The preferred embodiments of the present invention will be described below with reference to the accompanying drawings.

### First Embodiment

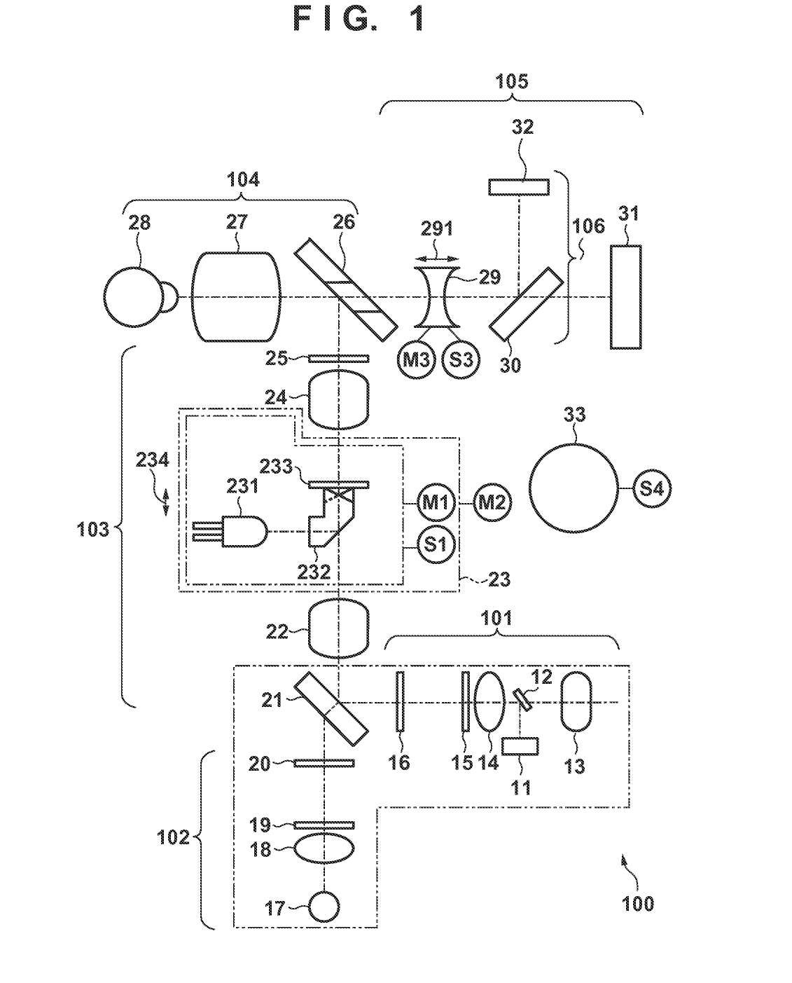

A fundus camera as an ophthalmic apparatus according to the first embodiment will be described with reference to FIGS. 1 to 9. The schematic arrangement of this camera will be described first with reference to FIG. 1. FIG. 1 is a schematic view for explaining the arrangement of the fundus camera according to the first embodiment.

A fundus camera 100 is roughly constituted by an imaging light source part 101, an observation light source part 102, an illumination optical system 103, an imaging/illumination optical system 104, an imaging optical system 105, and an internal fixation lamp part 106. The light beam emitted from the imaging light source part 101 or the observation light source part 102 illuminates a fundus portion of an object to be examined through the illumination optical system 103 and the imaging/illumination optical system 104. An image of the fundus portion is formed on an image sensor 31 through the imaging/illumination optical system 104 and the imaging optical system 105.

The imaging light source part 101 generates ring illumination of white light with the following arrangement. In the imaging light source part 101, reference numeral 11 denotes a light amount detection part which is a sensor using a known photoelectric conversion element such as a silicon photocell (SPC) or photodiode (PD); 12, a mirror which is formed by depositing aluminum or silver on a glass plate or from an aluminum plate or the like; and 13, an imaging light source which is an illumination light source for illuminating the fundus for imaging. As the imaging light source 13, for example, a xenon lamp with xenon (Xe) being sealed in a glass tube is used. The imaging light source 13 can obtain white light with a sufficient intensity for recording a fundus image at the time of imaging by emitting light upon application of a voltage. Reference numeral 14 denotes an imaging condenser lens which is a general spherical lens; 15, an imaging ring slit which is a flat plate having an annular opening; and 16, an imaging crystalline lens baffle which is also a flat panel having an annular opening. The imaging condenser lens 14 condenses a light beam toward the fundus of the eye to be examined. The imaging ring slit 15 then forms the light beam into an annular shape when it passes through the anterior ocular segment of the eye. The imaging crystalline lens baffle 16 limits a light beam projected on the crystalline lens of the eye to prevent unnecessary reflected light from the crystalline lens of the eye from being depicted in a fundus image.

The observation light source part 102 generates ring illumination of infrared light with the following arrangement. In the observation light source part 102, reference numeral 17 denotes an observation light source which is an illumination light source used for observing the fundus. This is a light source capable of continuously emitting light like a halogen lamp or LED and outputs infrared light depending on the characteristic of the device or filter. In this specification, an illumination light source is a light source which illuminates the fundus of the eye to be examined and is a generic term of the imaging light source 13 and the observation light source 17. Reference numeral 18 denotes an observation condenser lens which is a general spherical lens; 19, an observation ring slit which is a flat plate having an annular opening; and 20, an observation crystalline lens baffle which is also a flat panel having an annular opening. These components are similar to those of the imaging light source part 101 except that these light sources differ in type. In the observation light source part 102, the observation condenser lens 18 condenses the light output from the observation light source 17, and the observation ring slit 19 shapes the light beam at the anterior ocular segment. The observation crystalline lens baffle 20 prevents reflected light from the crystalline lens from being depicted in a fundus image.

The illumination optical system 103 relays the light beam generated by the imaging light source part 101 and the observation light source part 102 and generates index images for focusing a fundus image. In the illumination optical system 103, reference numeral 21 denotes a dichroic mirror which transmits infrared light and reflects visible light. The dichroic mirror 21 reflects the light beam of visible light generated by the imaging light source part 101, and transmits the light beam of infrared light generated by the observation light source part 102. Each light beam is then guided to the illumination optical system 103. Reference numeral 22 denotes a first illumination relay lens; and 24, a second illumination relay lens. These lenses form ring illumination into an image on the eye.

Reference numeral 23 denotes a split unit which includes a focus index light source 231 for projecting focus indices, a prism 232 for splitting light emitted by the focus index light source 231, and a focus index mask 233 indicating the outer shapes of focus indices. The split unit 23 includes a moving mechanism for shifting/moving the focus indices in the optical axis direction by moving the focus index light source 231, the prism 232, and the focus index mask 233 in an arrow direction 234 in FIG. 1. The split unit 23 further includes an entering/retreating mechanism which causes the split unit 23 to enter the optical path of the illumination optical system 103 and to retreat from it. The moving mechanism includes a split shift driving motor M1 and a split position sensor S1, shifts the split unit 23 to focus on each focus index, and detects the stop position. The entering/retreating mechanism includes a split entering/retreating driving motor M2 which causes the split unit 23 to enter/retreat with respect to the optical path of the illumination optical system 103. The entering/retreating mechanism causes the split unit 23 to enter the optical path of the illumination optical system 103 to project split indices in an observation image at the time of fundus observation. At the time of fundus imaging, the entering/retreating mechanism causes the split unit 23 to retreat from the optical path of the illumination optical system 103 so as to prevent each focus index from being depicted in a captured image. Reference numeral 25 denotes a cornea baffle which prevents unnecessary reflected light from the cornea of the eye to be examined from being depicted in a fundus image.

The imaging/illumination optical system 104 projects an illumination light beam on the fundus of an eye 28 to be examined and guides a fundus image of the eye to be examined. In the imaging/illumination optical system 104, reference numeral 26 denotes a perforated mirror whose peripheral portion is a mirror and central portion is a hole. The light beam guided from the illumination optical system 103 is reflected by the mirror portion of the perforated mirror 26 and illuminates the fundus of the eye to be examined through an objective lens 27. The illuminated fundus image of the eye to be examined returns to the objective lens 27 and is guided to the imaging optical system 105 through the hole in the central portion of the perforated mirror 26.

The imaging optical system 105 forms a fundus image of the eye to be examined on the image sensor upon focus adjustment. In the imaging optical system 105, reference numeral 29 denotes a focus lens which is a lens for focus adjustment of an imaging light beam passing through the central hole of the perforated mirror 26 by moving in an arrow direction 291 in FIG. 1. Reference symbol M3 denotes a focus lens driving motor; S3, a focus lens position sensor which performs focusing by driving the focus lens 29 and detects its stop position. Reference numeral 31 denotes an image sensor which photoelectrically converts imaging light. A processing circuit (not shown) A/D-converts the electrical signal obtained by the imaging element 31 into digital data. A display device (not shown) then displays the data at the time of observation with infrared light. This signal is stored in a recording medium (not shown) at the time of imaging.

In the internal fixation lamp part 106, a half mirror 30 branches an optical path from the imaging optical system 105, and an internal fixation lamp unit 32 faces the optical path. The internal fixation lamp unit 32 is constituted by a plurality of LEDs and turns on an LED at a position corresponding to the visual fixation part selected by the examiner using a fixation lamp position designation member 66. By letting the object fix his/her vision to the turned-on LED, the examiner can obtain a fundus image in a desired direction.

The above schematic arrangement is held in one housing to form a fundus camera optical part. The fundus camera optical part is mounted on a sliding base (not shown) to allow positioning with the eye 28. The examiner operates a focusing operation member 33 to position the fundus camera optical part. A focusing operation member position sensor S4 can detect the operation position of the focusing operation member 33.

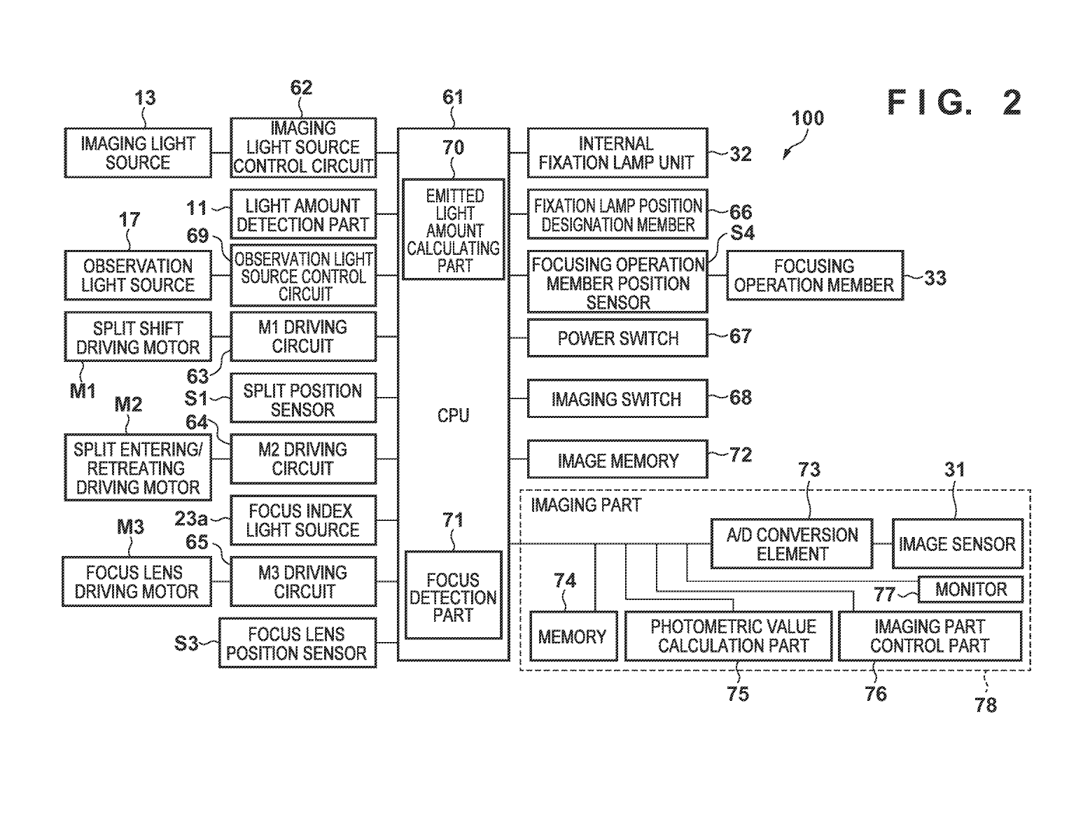

The above description is about the schematic arrangement using FIG. 1. The control arrangement of the fundus camera 100 will be described next with reference to FIG. 2. FIG. 2 is a block diagram for explaining the control arrangement of the fundus camera 100 according to the first embodiment.

A CPU 61 controls the following operation of the fundus camera 100. An imaging light source control circuit 62 charges energy for the emission of light by the imaging light source 13 before imaging. The imaging light source control circuit 62 discharges charged electric energy at the time of imaging to cause the imaging light source 13 to emit light. The light amount detection part 11 detects the emitted light amount of the imaging light source 13, and issues an instruction to stop light emission to the CPU 61 when the emitted light amount of the imaging light source 13 reaches the emitted light amount limited by an emitted light amount calculating part 70. Upon receiving the instruction to stop light emission from the light amount detection part 11, the CPU 61 stops light emission from the imaging light source 13 via the imaging light source control circuit 62. Both the imaging light source control circuit 62 connected to the imaging light source 13 and an observation light source control circuit 69 connected to the observation light source 17 are connected to the CPU 61 which also serves as the emitted light amount calculating part 70, thereby performing control such as light amount adjustment and ON/OFF control for the imaging light source 13 and the observation light source 17 as described above. An M2 driving circuit 64 drives the split entering/retreating driving motor M2 so as to cause the split unit 23 to enter/retreat with respect to the illumination optical system 103 before and after imaging. A power switch 67 is a switch for selecting power supply state for the fundus camera. An imaging switch 68 is a switch for executing imaging by the fundus camera.

When the examiner operates the focusing operation member 33, the focusing operation member position sensor S4 can detect the stop position of the focusing operation member 33. An M1 driving circuit 63 drives a split shift driving motor M1 to move the split to a position corresponding to an output from the focusing operation member position sensor S4 under the control of the CPU 61. Like the M1 driving circuit 63, an M3 driving circuit 65 drives the focus lens driving motor M3 to move the focus lens 29 to a position corresponding to an output from the focusing operation member position sensor S4 under the control of the CPU 61. In the manual focusing mode, the CPU 61 controls the split shift driving motor M1 and the focus lens driving motor M3 in accordance with outputs from the focusing operation member position sensor S4, as described above. In the autofocus mode, the CPU 61 controls the focus lens driving motor M3 via the M3 driving circuit 65 based on a detection result from a focus detection part 71 inside the CPU 61. That is, the fundus camera 100 of this embodiment has an autofocus function of automatically executing focus adjustment.

In an imaging part 78, an A/D conversion element 73 converts an output from the image sensor 31 into a digital signal, which is stored in a memory 74 and output to a photometric value calculation part 75. Note that the A/D conversion element 73, the memory 74, and the photometric value calculation part 75 are connected to the CPU 61. An image memory 72 is connected to the CPU 61. The image memory 72 stores the still image captured by the image sensor 31 as a digital image.

The imaging part 78 includes a monitor 77 for displaying the infrared observation image, visible captured image, and the like captured by the image sensor 31 and an imaging part control part 76, in addition to the image sensor 31, the A/D conversion element 73, the memory 74, and the photometric value calculation part 75. The imaging part 78 is detachably fixed to the housing of the fundus camera optical part with a mount portion (not shown). An electrical block will be described below with reference to FIG. 2.

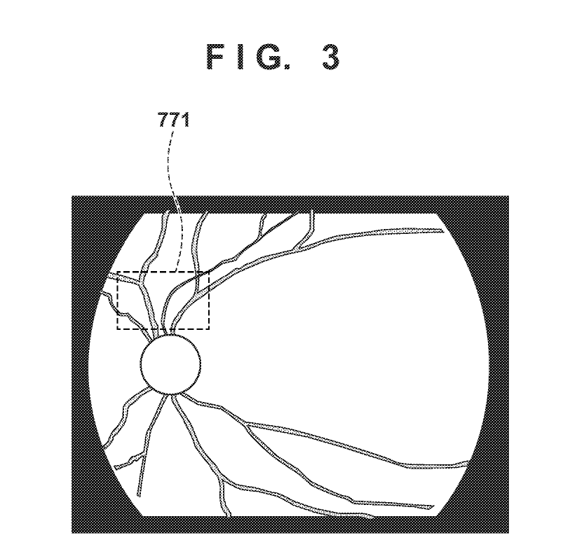

The fundus image of the eye to be examined which is displayed on the monitor 77 will be described next with reference to FIG. 3. FIG. 3 is a view showing the the fundus image of the eye to be examined and a focus detection range 771 which are displayed on the monitor 77 of the fundus camera 100 according to the first embodiment.

At the time of fundus observation, the apparatus presents a frame indicating the focus detection range 771 to the examiner upon superimposing it on the fundus image obtained by the imaging part 78. This makes it possible to visually present the focus detection position to the examiner, thereby improving the operability in autofocus. Note that the examiner can manually change the focus detection range, and may set a specific region on the fundus of the eye to be examined or the overall fundus as a focus detection range. The fundus image of the eye to be examined which is displayed on the monitor 77 has been described above with reference to FIG. 3.

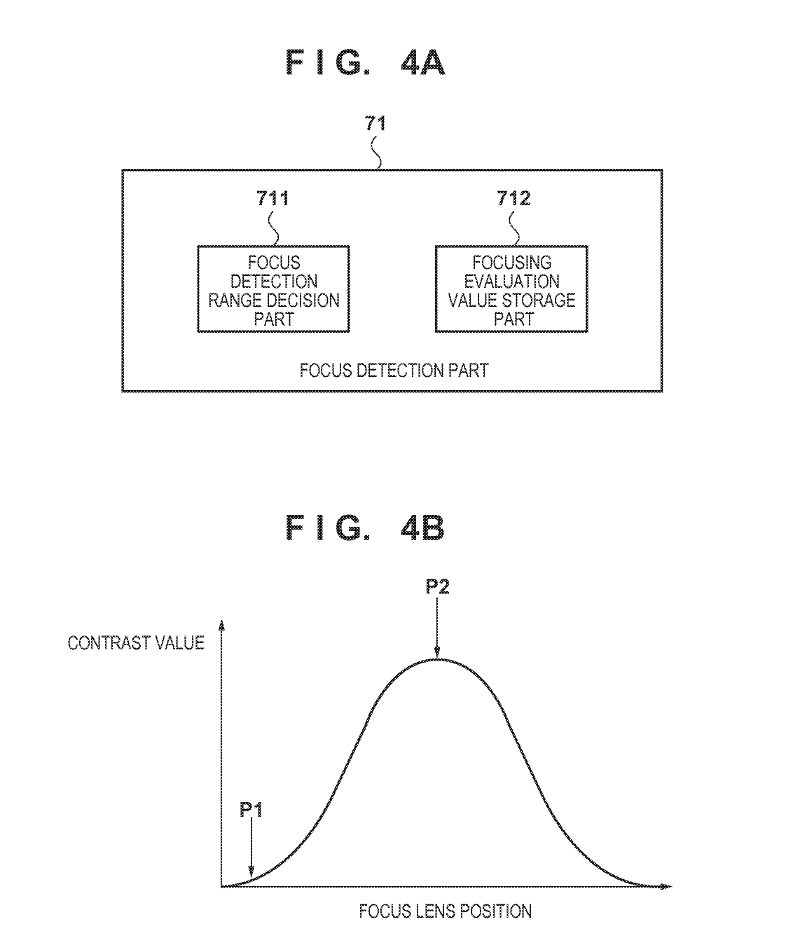

The schematic arrangement of the focus detection part 71 and the principle of contrast detection will be described next with reference to FIGS. 4A and 4B. FIG. 4A shows the schematic arrangement of the focus detection part 71 according to the first embodiment. FIG. 4B shows the principle of contrast detection according to this embodiment. Assume that the embodiment uses the luminance differences between adjacent pixels as contrasts and uses the largest luminance difference value among luminance data in a predetermined range as a contrast value. Note however that it is also possible to use, as a contrast value, a value other than the largest luminance difference value among luminance data in a predetermined range.

As shown in FIG. 4A, the focus detection part 71 is provided with a focus detection range decision part 711 which sets a specific position on the fundus of the eye 28 as a focus detection target. The examiner can decide the focus detection range 771 by operating the operation input part. In addition, the focus detection part 71 incorporates a focusing evaluation value storage part 712 which stores the contrast values of a fundus image and the positions of the focus lens 29. This embodiment performs focus detection by detecting the contrast value of the fundus image itself which is formed by an imaging light beam.

The graph of FIG. 4B represents the contrast value transition relative to the position of the focus lens 29 moved by the focus lens driving motor M3. As is obvious from FIG. 4B, the contrast value is maximized at a focal position P2, whereas the contrast value is reduced at a position P1 where the amount of defocusing is large. This embodiment can perform focus detection without influence of aberration of the eye to be examined by using this principle of contrast detection. This is because the position of the focus lens 29 moved to the focal position P2 by the focus lens driving motor M3 coincides with:
the position where the examiner can observe the fundus image displayed on the monitor 77 most clearly; and
the position of the focus lens 29 at which he fundus image displayed on the monitor 77 after imaging can be made most clearly. The schematic arrangement of the focus detection part 71 and the principle of contrast detection have been described above with reference to FIGS. 4A and 4B.

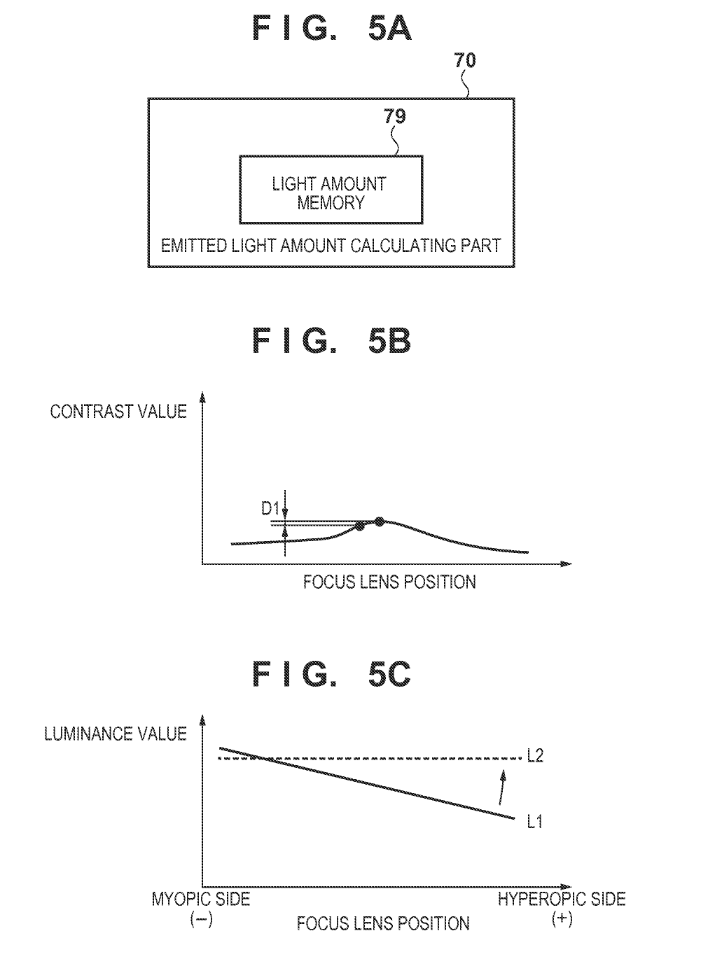

The emitted light amount calculating part 70 will be described next with reference to FIGS. 5A to 5C. FIG. 5A shows the schematic arrangement of the emitted light amount calculating part 70 according to the first embodiment. FIG. 5B is a graph showing a concept of contrast value transition in infrared light. FIG. 5C is a graph showing a concept of luminance value transition on the image sensor 31 corresponding to focus lens positions.

The A/D conversion element 73 A/D-converts an output from each pixel of the image sensor 31. The memory 74 temporarily stores the digital data. The photometric value calculation part 75 outputs, as a photometric value, the maximum value of the luminance values in a focus detection range from the pixel outputs stored in the memory 74 to the emitted light amount calculating part 70. However, the acquisition of a photometric value is not limited to this. For example, a dedicated actinometer may he placed to measure the amount of reflected light from the fundus. As shown in FIG. 5A, the emitted light amount calculating part 70 includes a light amount memory 79 which stores a reference value for an observation light amount which is determined suitably for focus detection, and decides the emitted light amount of observation light by comparing a photometric value with the reference value.

If, for example, a photometric value is larger than the reference value, the emitted light amount calculating part 70 determines that the amount of observation light illuminating the fundus is large, and decides an emitted light amount so as to reduce the light amount to prevent luminance value saturation. In contrast, if a photometric value is smaller than the reference value, the emitted light amount calculating part 70 determines that the amount of observation light illuminating the fundus is small, and decides an emitted light amount to increase the light amount to facilitate detection of a maximal point of contrast values.

Contrast value transition when the contrast value of a fundus image of the eye to be examined is calculated by using infrared light will be described below. FIG. 5B shows contrast value transition relative to the position of the focus lens 29 moved by the focus lens driving motor M3 when infrared light is used as observation light. Although the description with reference FIG. 4B uses a graph or ideal contrast value transition for the explanation of the principle of contrast detection, a contrast value difference D1 in FIG. 5B is smaller than that in the graph of FIG. 4B. In addition, as indicated by a solid line L1 in the graph of FIG. 5C, as the position of the focus lens shifts to the hyperopic side in terms of focus, the luminance value captured by the image sensor 31 decreases. As the luminance value decreases and the contrast value difference D1 decreases, it becomes more difficult to detect the peak of contrast values. This makes it difficult to perform accurate focusing.

The light amount memory 79 incorporated in the emitted light amount calculating part 70 stores both the reference value for observation light amounts which is determined suitably for focus detection and luminance variation values on the image sensor 31 which correspond to the positions of the focus lens 29. The emitted light amount calculating part 70 decides the emitted light amount of observation light by comparing the photometric value calculated by the photometric value calculation part 75 with the reference value for observation light amounts which is stored in the light amount memory 79. In this case, the emitted light amount calculating part 70 decides the emitted light amount of observation light corresponding to the focus lens position by using the luminance variation value on the image sensor 31 corresponding to the position of the focus lens 29 which is stored in the light amount memory 79. That is, the emitted light amount calculating part 70 changes the amount of observation light in accordance with the position of the focus lens. For example, the CPU 61 adjusts the emitted light amount of observation light by the observation light source 17 to match the luminance value indicated by a dotted line L2 in FIG. 5C, thereby canceling out variations corresponding to the positions of the focus lens.

Although variation values are stored in the light amount memory 79 in advance, the present invention is not limited to this. For example, the apparatus may measure the luminance on the image sensor 31 after the movement of the focus lens 29, compare the measured luminance with the reference value stored in the light amount memory, and store the difference between the measured luminance and the reference value in the light amount memory 79. That is, the apparatus may store the difference between the reference value and the luminance measured in real time in the light amount memory 79 and change the observation light amount based on the difference between the reference value and the measured and stored luminance.

Although this embodiment uses infrared light as observation light, the present invention is not limited to this. Even when calculating the contrast value of a fundus image of the eye to be examined by using visible light, the apparatus may change the emitted light amount of observation light in accordance with the focus lens position in the same manner as described above.

In this embodiment, the light amount memory 79 stores both the reference value for observation light amounts and luminance variation values on the image sensor 31 which correspond to the positions of the focus lens 29. However, the present invention is not limited to this. The light amount memory 79 may store only luminance variation values on the image sensor 31 which correspond to the positions of the focus lens 29, and the emitted light amount calculating part 70 may decide the emitted light amount of observation light corresponding to the focus lens position by using such a variation value.

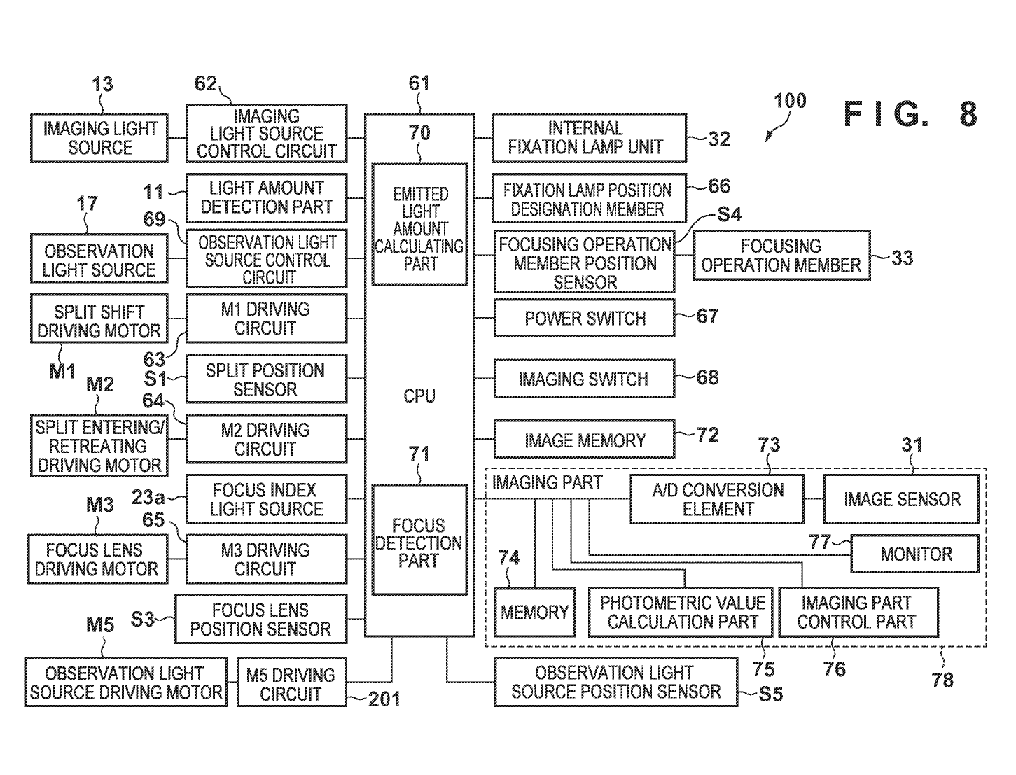

This embodiment is configured to set luminance values like those on the dotted line L2 shown in FIG. 8 by controlling the emitted light amount of observation light from the observation light source 17 in accordance with variations in luminance values on the image sensor 31 in correspondence with the positions of the focus lens. However, the present invention is not limited to this. For example, the imaging part control part 76 may adjust the gain of the image sensor 31 in accordance with the luminance variation values on the image sensor 31 which are stored in the light amount memory 79 in accordance with the positions of the focus lens 29. For example, it is possible to implement this operation by making the emitted light amount calculating part 70 inform the imaging part control part 76 of variation values corresponding to the positions of the focus lens 29 and then making the imaging part control part 76 control the gain of the image sensor 31 in accordance with the variation values. In this case, for example, the apparatus obtains values which almost match the luminances of the images at the respective positions of the focus lens 29 from the images obtained in advance upon moving the focus lens 29, and uses such values as variation values. The apparatus then stores the positions of the focus lens 29 and variation values in the light amount memory 79 in correspondence with each other.

The apparatus may offset the contrast value which is stored in the focusing evaluating value storage part 712 and makes transition in accordance with the focus lens position based on the variation values stored in the light amount memory 79 instead of controlling the emitted light amount of the observation light source 17 or the gain of the image sensor 31 in the above manner. In this case, the emitted light amount calculating part 70 informs the focus detection part 71 of a luminance variation value on the image sensor 31 which is stored in the light amount memory 79 in correspondence with the position of the focus lens 29. The focus detection part 71 offsets the contrast value stored in the focusing evaluation value storage part 712 based on the informed variation value. For example, it is possible to change the contrast value by performing tone conversion for the obtained image. In this case, for example, the apparatus obtains a tone conversion characteristic which almost matches the luminances of the images at the respective positions of the focus lens 29 from the images obtained in advance upon moving the focus lens 29. The apparatus then stores these tone conversion characteristics in the light amount memory 79 in correspondence with the positions of the focus lens 29 and the variation values. Note that it is possible to combine some or all of the above control schemes, that is, control on the emitted light amount of the observation light source 17, control on the gain of the image sensor 31, and offset control on contrast values.

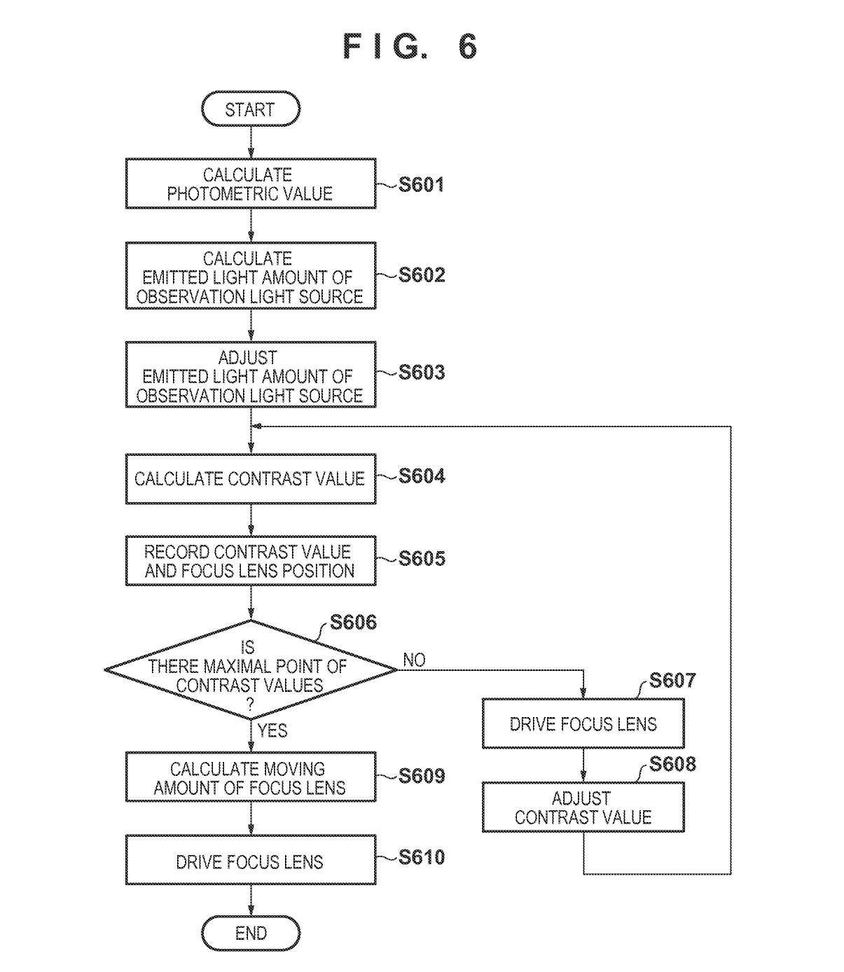

The emitted light amount calculating part 70 have been described above with reference to FIGS. 5A to 5C. Autofocus processing by the fundus camera 100 according to this embodiment will be described next with reference to FIG. 6.

When the examiner issues an instruction to start autofocus, the photometric value calculation part 75 calculates the maximum value of luminance values in a focus detection range as a photometric value from the pixels stored in the memory 74, and outputs the photometric value to the emitted light amount calculating part 70 in step S601. In step S602, the emitted light amount calculating part 70 compares the reference value for emitted light amounts which is stored in the light amount memory 79 with the photometric value calculated in step S601 to decide the emitted light amount of observation light from the observation light source 17 so as to match, for example, the photometric value with the reference value. In addition, in step S602, the emitted light amount calculating part 70 compares the photometric value calculated in step S601 with the luminance variation value on the image sensor 31 which is stored in the light amount memory 79 in correspondence with the position of the focus lens 29. The emitted light amount calculating part 70 then decides the emitted light amount of the observation light source 17 so as to compensate for the variation value corresponding to the position of the focus lens 29. In step S603, the emitted light amount calculating part 70 controls the observation light source control circuit 69 so as to irradiate the fundus with the amount of observation light decided in step S602. That is, the emitted light amount calculating part 70 controls the observation light amount so as to match the photometric value with the reference value stored in the light amount memory 79. With this operation, the apparatus irradiates the fundus with observation light decided in step S602 in accordance with the position of the focus lens 29.

In step S604, the focus detection part 71 calculates a contrast value based on the image obtained from the imaging part 78. In step S605, the focus detection part 71 records the contrast value calculated in step S604 and the position of the focus lens 29 on the focusing evaluation value storage part 712. In step S606, the focus detection part 71 detects whether the contrast values recorded on the focusing evaluation value storage part 712 in step S605 include a maximal point like the position P2 shown in FIG. 4B.

If the focus detection part 71 does not detect any maximal point in step S606, the process advances to step S607, in which the CPU 61 changes the focus lens position by driving the focus lens 29 by a predetermined moving amount. In step S608, the CPU 61 adjusts the emitted light amount of observation light from the observation light source 17 based on the relationship between focus lens positions and luminance values, as shown in FIG. 5C, and adjusts the contrast value by canceling out the variation corresponding to the position of the focus lens. The process then returns to the processing in step S604. Subsequently, the CPU 61 repeats steps S607, S608, S604, and S605 until the detection of a maximal point of contrast values in step S606.

If the focus detection part 71 detects a maximal point in step S606, the process advances to step S609. In step S609, the focus detection part 71 calculates the moving amount of the focus lens 29. In this case, the moving amount of the focus lens 29 is the driving amount of the focus lens to the detection position of the maximal point. In step S610, the CPU 61 drives the focus lens 29 in accordance with the moving amount of the focus lens 29 calculated in step S609 to move the position of the focus lens 29 to the position of the maximal value of contrast values. With the above operation, even if eyes 28 of different objects have individual differences in aberrations such as aspherical aberration and astigmatism, it is possible to perform focus adjustment in accordance with such aberrations. Note that the emitted light amount calculating part 70 controls the emitted light amount of the imaging light source 13 based on the observation light amount variation value at the position of the focus lens 29 calculated in step S609. For example, the emitted light amount calculating part 70 changes the emitted light amount of the imaging light source 13 by the observation light amount variation value at the position of the focus lens 29. Note that it is possible to obtain the observation light amount variation value at the position of the focus lens 29 from the variation values stored in the light amount memory 79 by using an approximate expression. The apparatus then performs imaging with this controlled light amount.

The above autofocus operation is especially effective in a non-mydriatic fundus camera designed to perform observation by using infrared light. Since the contrast of medium and large vessels on the fundus is low relative to infrared light, a contrast value difference is difficult to appear with respect to focus lens positions. It is therefore difficult to detect the position P2 corresponding to the maximal point shown in FIG. 4B in autofocus. It is therefore necessary to increase the emitted light amount of the infrared LED for illuminating the fundus to increase the contrast of an observation image as much as possible. If, however, the fundus becomes brighter than necessary, luminance value saturation occurs, leading to the failure to correctly calculate a contrast value. In contrast to this, the fundus camera 100 according to this embodiment prevents luminance value saturation in advance by calculating and controlling a proper observation light amount before the calculation of a contrast value, and corrects an observation light amount with respect to the luminance value which varies in accordance with the position of the focus lens 29. This makes it possible to stably calculate a contrast value and allows to perform accurate focus detection.

### Second Embodiment

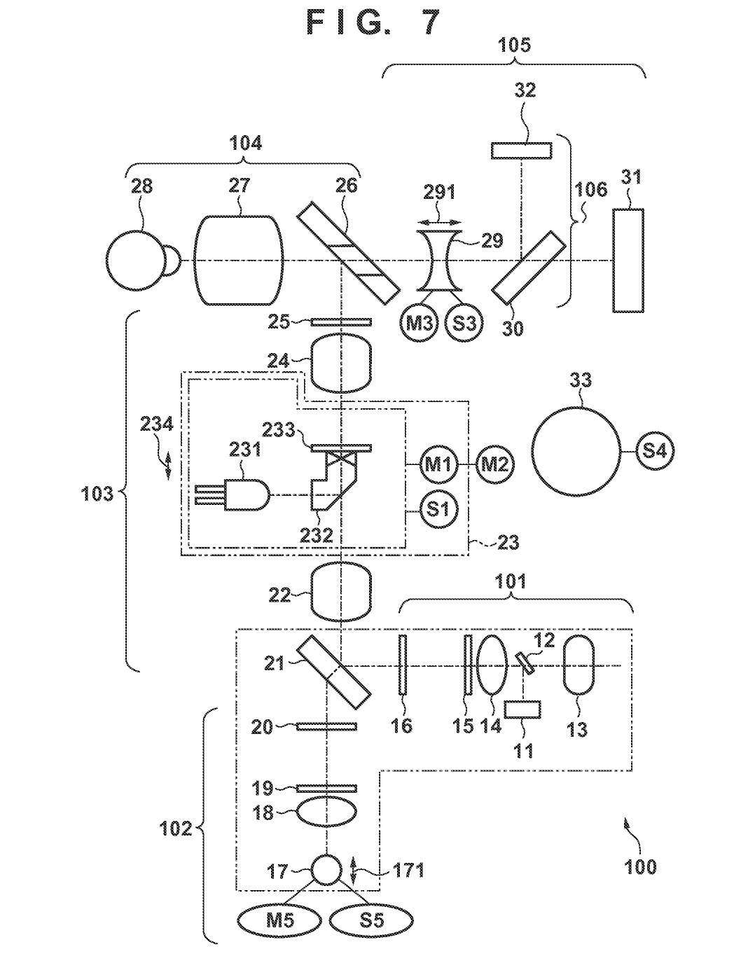

A fundus camera 100 according to the second embodiment will be described in detail next with reference to FIGS. 7 to 9. The first embodiment has exemplified the arrangement for removing the influence of variations (FIG. 5C) in luminance value in accordance with focus lens positions by adjustment on the emitted light amount of the observation light source 17, and adjustment of the gain of the image sensor 31, and/or offset adjustment on measured contrast values. The second embodiment is configured to remove the influence of variations in luminance value (FIG. 5C) in accordance with focus lens positions by moving an observation light source 17.

The fundus camera 100 according to the second embodiment is configured to move the observation light source 17 in the direction indicated by an arrow 171 in FIG. 7 relative to an observation light source part 102 in accordance with the luminance variation value on an image sensor 31 which is stored in a light amount memory 79 in an emitted light amount calculating part 70 in correspondence with the position of the focus lens 29. This controls the amount of observation light which irradiates the fundus of the eye to be examined. When increasing the amount of observation light, the fundus camera 100 moves the observation light source 17 in the direction to approach the eye to be examined. When decreasing the amount of observation light, the fundus camera 100 moves the observation light source 17 in the direction to separate from the eye.

The schematic arrangement of the fundus camera 100 according to the second embodiment will be described with reference to FIG. 7. The fundus camera 100 according to the second embodiment includes a mechanism for moving the observation light source 17 in the direction indicated by the arrow 171 in FIG. 7, in addition to the arrangement of the first embodiment (FIG. 1), in order to control the amount of observation light which irradiates the fundus of an eye 28 to be examined. Reference symbol M5 denotes an observation light source driving motor which moves the observation light source 17 in the direction indicated by the arrow 171; S5, an observation light source position sensor which detects the stop position of the observation light source 17 moved by the observation light source driving motor M5. Other arrangements are the same as those described in the first embodiment (FIG. 1).

The control arrangement of the fundus camera 100 according to the second embodiment will be described next with reference to FIG. 8. FIG. 8 is a block diagram showing the control arrangement of the fundus camera 100 according to the second embodiment. In addition to the control arrangement (FIG. 2) described in the first embodiment, the control arrangement of the fundus camera 100 according to the second embodiment includes an M5 driving circuit 201 controlled by a CPU 61, the observation light source driving motor M5, and the observation light source position sensor S5.

The M5 driving circuit 201 drives the observation light source 17 via the observation light source driving motor M5 based on the luminance variation value on the image sensor 31 which is stored in the light amount memory 79 incorporated in the emitted light amount calculating part 70 in accordance with the position of a focus lens 29. The observation light source position sensor S5 is based on an output from a focus lens position sensor S3. The observation light source 17 is driven in accordance with the position of the focus lens 29. Other components are the same as those of the control arrangement (FIG. 2) described in the first embodiment of the present invention.

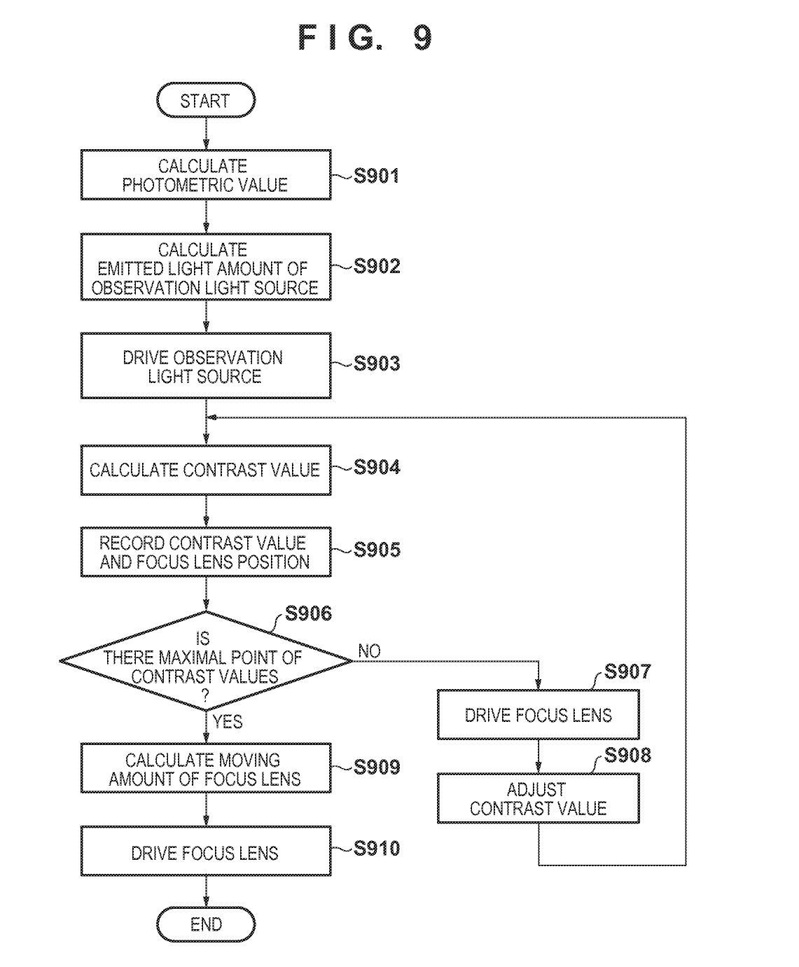

Autofocus processing by the fundus camera 100 according to the second embodiment will be described next with reference to the flowchart of FIG. 9.

When the examiner issues an instruction to start autofocus, a photometric value calculation part 75 calculates the maximum value of luminance values in a focus detection range as a photometric value from the pixel outputs stored in a memory 74, and outputs the photometric value to the emitted light amount calculating part 70 in step S901. In step S902, the emitted light amount calculating part 70 compares the reference value for emitted light amounts which is stored in the light amount memory 79 with the photometric value calculated in step S901 to decide the emitted light amount of the observation light source 17 so as to match the photometric value with the reference value. In addition, in step S902, the emitted light amount calculating part 70 compares the photometric value calculated in step S901 with the luminance variation value on the image sensor 31 which is stored in the light amount memory 79 in correspondence with the position of the focus lens 29. The emitted light amount calculating part 70 then decides the position of the observation light source so as to compensate for the variation value corresponding to the position of the focus lens 29 based on this comparison result. In step S903, the CPU 61 controls an observation light source control circuit 69 to irradiate the fundus with the amount of observation light decided in step S902. In step S903, the CPU 61 drives the observation light source driving motor M5 via the M5 driving circuit 201 to move the observation light source to the position decided in step S902 which corresponds to the position of the focus lens 29.

In step S904, the focus detection part 71 calculates a contrast value. In step S905, the focus detection part 71 records the contrast value calculated in step S904 and the position of the focus lens 29 on a focusing evaluation value storage part 712. In step S906, the focus detection part 71 detects whether the contrast values recorded in step S905 include a maximal point like the position P2 shown in FIG. 4B.

If the focus detection part 71 does not detect any maximal point in step S906, the process advances to step S907, in which the focus detection part 71 changes the focus lens position by driving the focus lens 29 by a predetermined moving amount. In step S908, the focus detection part 71 adjusts the contrast value. More specifically, the M5 driving circuit 201 drives the observation light source 17 via the observation light source driving motor M5 in accordance with the amount of variation in luminance value in accordance with the position of the focus lens 29, an output from the focus lens position sensor S3, and an output from the observation light source position sensor S5. In this manner, the apparatus performs control to place the observation light source 17 at a position to cancel out the amount of variation in luminance value at the position of the focus lens 29. Subsequently, the apparatus repeats steps S907, S908, S904, and S905 until the detection of a maximal point of contrast values in step S906.

If the focus detection part 71 detects a maximal point in step S906, the process advances to step S909. In step S909, the focus detection part 71 calculates the moving amount of the focus lens 29. In this case, the moving amount of the focus lens 29 is the driving amount of the focus lens to the detection position of the maximal point. In step S910, the focus detection part 71 drives the focus lens 29 in accordance with the moving amount of the focus lens 29 calculated in step S909 to move the focus lens 29 to the position of the maximal value of contrast values. With the above operation, even if eyes 28 of different objects have individual differences in aberrations such as aspherical aberration and astigmatism, it is possible to perform focus adjustment in accordance with such aberrations. Note that in this case, as in the first embodiment, the emitted light amount calculating part 70 may control the emitted light amount of the imaging light source 13 based on the observation light amount variation value at the position of the focus lens 29 calculated in step S909.

As in the first embodiment, the above operation is especially effective in a non-mydriatic fundus camera designed to perform observation by using infrared light. Since the contrast of medium and large vessels on the fundus is low relative to infrared light, a contrast value difference is difficult to appear with respect to focus lens positions. It is therefore difficult to detect the position P2 corresponding to the maximal point shown in FIG. 4B in autofocus. It is therefore necessary to increase the emitted light amount of the infrared LED for illuminating the fundus to increase the contrast of an observation image as much as possible. If, however, the fundus becomes brighter than necessary, luminance value saturation occurs, leading to the failure to correctly calculate a contrast value. In contrast to this, the fundus camera 100 according to the second embodiment prevents luminance value saturation in advance by calculating and controlling a proper observation light amount (emitted light amount) before the calculation of a contrast value, and corrects an observation light amount with respect to the luminance value which varies in accordance with the position of the focus lens 29 by changing the position of the observation light source 17. This makes it possible to stably calculate a contrast value and allows to perform accurate focus detection.

Aspects of the present invention can also be realized by a computer of a system or apparatus (or devices such as a CPU or MPU) that reads out and executes a program recorded on a memory device to perform the functions of the above-described embodiment(s), and by a method, the steps of which are performed by a computer of a system or apparatus by, for example, reading out and executing a program recorded on a memory device to perform the functions of the above-described embodiment(s). For this purpose, the program is provided to the computer for example via a network or from a recording medium of various types serving as the memory device (for example, computer-readable storage medium).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-237265, filed Oct. 26, 2012, which is hereby incorporated by reference herein in its entirety.

What is claimed is:

1. An ophthalmic apparatus comprising:
an illumination optical system which projects an illumination light beam from an illumination light source onto a fundus of an eye to be examined;
an imaging optical system which guides reflected light from the fundus to imaging unit;
a calculation unit configured to calculate a contrast value of a fundus image formed by said imaging unit;
a focusing unit configured to focus said imaging optical system on the fundus by moving a focus lens in an optical-axis direction of said imaging optical system based on a contrast value obtained by said calculating unit; and
an adjusting unit configured to adjust the contrast value obtained by said calculating unit based on a position of the focus lens in the optical-axis direction.

2. The apparatus according to claim 1, wherein said adjusting unit adjusts the contrast value by controlling a light amount of the illumination light source in accordance with the position of the focus lens.

3. The apparatus according to claim 1, wherein said adjusting unit adjusts the contrast value by controlling a gain of said imaging unit in accordance with the position of the focus lens.

4. The apparatus according to claim 1, wherein said adjusting unit adjusts the contrast value by changing an offset to be provided for the contrast value in accordance with the position of the focus lens.

5. The apparatus according to claim 1, wherein the illumination light source is provided to be configured to move in the optical-axis direction of said illumination optical system, and
said adjusting unit adjusts the contrast value by moving the illumination light source in accordance with the position of the focus lens.

6. The apparatus according to claim 1, further comprising a photometric unit configured to photometrically measure the reflected light from the fundus,
wherein said adjusting unit adjusts the light amount of the illumination light source based on a photometric value obtained by said photometric unit.

7. The apparatus according to claim 6, wherein said photometric unit acquires, as the photometric value, a maximum luminance value from a specific range of an image obtained from said imaging unit.

8. A control method for an ophthalmic apparatus including an illumination optical system which projects an illumination light beam from an illumination light source onto a fundus of an eye to be examined and an imaging optical system which guides reflected light from the fundus to imaging unit, the method comprising:
a calculation step of calculating a contrast value of a fundus image formed by the imaging unit;
a focusing step of focusing the imaging optical system on the fundus by moving a focus lens in an optical-axis direction of the imaging optical system based on a contrast value obtained in the calculation step; and
an adjusting step of adjusting the contrast value obtained in the calculation step based on a position of the focus lens in the optical-axis direction.

9. A non-transitory computer-readable storage medium storing a program for causing a computer to execute each step in a control method for an ophthalmic apparatus defined in claim 8.
